(19) United States
(12) Patent Application Publication
(10) Pub. No.: US 2002/0115729 A1
(43) Pub. Date: Aug. 22, 2002

(54) Stabilization method and composition utilizing an amphoteric polymer

(76) Inventor: Robert K. Yang, Flushing, NY (US)

Correspondence Address: CHRISTIE, PARKER & HALE, LLP, 350 WEST COLORADO BOULEVARD, SUITE 500, PASADENA, CA, 91105, US

(21) Appl. No.: 09/995,326

(22) Filed: Nov. 27, 2001

Related U.S. Application Data
(60) Provisional application No. 60/253,598, filed on Nov. 27, 2000.
(60) Provisional application No. 60/253,599, filed on Nov. 27, 2000.

(51) Int. Cl. A61K 47/32; C12H 1/00; A23K 1/00; C12C 1/00; A23L 2/00; A23C 3/00; A23L 3/36; A21D 4/00; A23B 4/00

(52) U.S. Cl. 514/772.5; 426/330; 426/330.6

(57) ABSTRACT

A method for increasing the stability of a food-grade or pharmaceutical-grade liquid, such as an extracted oil, herbal extract, flavor, color, or volatile chemical component used in the flavor industry, is provided. The method comprises mixing the liquid with an amphoteric polymer, preferably polyvinylpyrrolidone, to thereby infuse the liquid into the amphoteric polymer matrix and form a generally-solid, stabilized product. Optionally, bulking agents, absorbents, and flowing agents can be mixed with the liquid and amphoteric polymer to enhance the properties of the stabilized product. The inventive method is particularly useful for entrapping liquids that are highly volatile, heat sensitive and/or easily oxidizable.

### CROSS-REFERENCE TO RELATED APPLICATIONS

This application claims the benefit of U.S. Provisional Patent Application No. 60/253,598, filed Nov. 27, 2000, and U.S. Provisional Patent Application No. 60/253,599, filed Nov. 27, 2000, the entire disclosures of which are incorporated herein by reference.

### BACKGROUND

It is often necessary to entrap food-grade or pharmaceutical-grade liquids that are either highly volatile or heat sensitive into polymeric substances. Examples of such liquids include dimethyl sulfide, acetaldehyde, mint flavors, butyric acid, and citrus flavors, which are known to be difficult to entrap in polymeric substances. Of particular interest are food-grade colored oleoresins, which are routinely used in various processed foods to supplement the freshness color of its ingredients, such as oleoresin paprika used in potatoes or oleoresin capsicum used in tomato sauce. Such oleoresins are often not stable, particularly in the presence of a salt solution, which is a strong oxidizing agent. Oxidized oleo-resins have a reduced ability to reflect colors, thereby reducing their effectiveness in food products.

Various methods exist for entrapping oleoresins and other unstable food flavors and pharmaceutical grade liquids in polymeric substances. Such methods include spray drying, fluidization, spray congealing, extrusion-dry and melt processes, and coacervation in aqueous and non-aqueous systems. However, all of these methods utilize heat or solvent, which can change the composition of the entrapped liquid. Furthermore, these methods disadvantageously permit only a limited amount of liquid to be entrapped in the polymeric substance. These methods can also involve costly equipment, further limiting their desirability.

Accordingly, a need exists for an alternative method for entrapping liquids that are highly volatile or heat sensitive into polymeric substances without exposing the liquid to heat. The present invention satisfies these and other needs and provides further related advantages.

### SUMMARY OF THE INVENTION

The invention is directed to novel methods for infusing food-grade and pharmaceutical-grade liquids into an amphoteric polymer matrix and the stabilized product thus produced. The invention is particularly useful for entrapping liquids that are highly volatile, heat sensitive and/or easily oxidizable.

In one embodiment, the invention is directed to a method for increasing the stability of a food-grade or pharmaceutical-grade liquid, such as an extracted oil, flavor, or color. The method comprises mixing the liquid with an amphoteric polymer, preferably polyvinylpyrrolidone, to thereby infuse the liquid into the amphoteric polymer matrix and form a generally-solid, stabilized product. Optionally, bulking agents, absorbents, and/or flowing agents can be mixed with the liquid and amphoteric polymer to enhance the properties of the stabilized product.

In another embodiment, the invention is directed to a stabilized generally-solid composition comprising an amphoteric polymer in which is infused a food-grade or pharmaceutical-grade liquid. The composition can optionally include bulking agents, absorbents, and/or flowing agents.

### DETAILED DESCRIPTION

The invention is directed to a method for increasing the stability of a food-grade or pharmaceutical-grade liquid comprising mixing the liquid with an amphoteric polymer to thereby entrap the liquid into a matrix of the amphoteric polymer. Advantageously, the liquid is infused into the polymer matrix, whereby the liquid and amphoteric polymer are physically compatible to entrap the liquid into the polymer matrix at a molecular level. The infused liquid remains entrapped in the polymer matrix until the matrix subsequently degenerates. Because of the polymer's amphoteric character, the liquid will be released from the polymer matrix when the amphoteric polymer is in the presence of sufficient moisture or exposed to thermal processing. The infusion process of the present invention does not employ heat or solvents to be evaporated. In addition, the methods of the invention do not employ expensive equipment. Accordingly, the inventive methods increase the shelf-life and cost-effectiveness of traditionally unstable liquids. The invention is also directed to a composition comprising a food-grade or pharmaceutical-grade liquid entrapped in an amphoteric polymer.

The present invention can be used in connection with any food-grade or pharmaceutical-grade liquids, such as such as extracted oils, herbal extracts, flavors, colors, and volatile chemical components used in the flavor industry, for which enhanced stability is desirable. The invention is particularly useful in connection with liquids that are heat-sensitive, highly volatile or easily oxidized.

Flavors may be chose from natural and synthetic flavoring liquids, such as, for example, volatile oils, synthetic flavor oils, flavoring aromatics, oleoresins, extracts derived from plants, leaves flowers, fruits and stems, and combinations thereof. More particular examples of suitable flavors suitable for use in the present invention include citrus oils, such as lemon, orange, grape, lime and grapefruit oils, and fruit essences, such as apple, pear, peach, grape, strawberry, raspberry, cherry, plum, pineapple, and apricot essences. Other useful flavorings include aldehydes and esters, such as benzaldehyde (cherry, almond), citral, i.e., alphacitral or beta-citral (lemon, lime), decanal (orange, lemon, aldehyde C-8 (citrus fruits), aldehyde C-9 (citrus fruits), aldehyde C-12 (citrus fruits), tolyl aldehyde (cherry, almond), 2,6-dimethyloctanol (green fruit) and 2-dodecenal (citrus, mandarin), and combinations thereof. Other flavors suitable for use in the invention include mint flavor, diacetyl flavor, mustard flavor, peppermint oil, lemon flavor, cheese flavor (such as butyric acid), acetaldehyde, onion oil, garlic oil, mustard flavor, smoke flavor, honey flavor, maple flavor, yeast dough flavor, cilantro flavor, tabasco flavor, worcestershire flavor, coffee flavor, and coconut flavor. Other liquids particular suitable for use in the invention include oleoresins, such as paprika, capsicum, black pepper, apsicum, basil, oregano, ginger, clove, curry blend, jalapeno, chipotle chili, cajun blend, cilantro, celery, chili spice, green bell pepper, barbecue blend allspice, and chili ancho. Examples of volatile chemical components used in the flavor industry include, for example, dimethyl sulfide, isobutyl aldehyde, 2-methyl-3-furanthiol, methional, acetaldehyde and sulfural.

Examples of extracted oils include fish-oils, garlic oil and onion oil. Herbal extracts suitable for use in the present invention include, but are not limited to, schizandrae berry, rehmannia, hawthorne berry, jujube, alisma, angelicae dahuricae, epemidium, piria cocos, rhizoma rhei, stephania root, angelicae sinensis root, codonoposis root, eucommium bark, notoginseng, echinacea, ginkgo biboba, St. John's wort, kava kava, aloe vera, chamomile, saw palmetto, and lemon balm.

The food-grade or pharmaceutical-grade liquid is preferably included in an amount ranging from about 3 wt. % to about 70 wt. %, more preferably from about 4 wt. % to about 50 wt. %, still more preferably from about 5 wt. % to about 30 wt. %, based on the total weight of the final stabilized product.

The invention can be used in connection with any suitable amphoteric polymer, which is a polymer that can be dissolved in both organic and aqueous solutions. A particularly preferred amphoteric polymer for use in connection with the invention is polyvinylpyrrolidone (PVP), such as Plastone USP grade K29-32 (commercially available from International Specialty Products, Wayne, N.J.). The amphoteric polymer is preferably included in an amount ranging from about 3 wt. % to about 40 wt. %, more preferably from about 4 wt. % to about 30 wt. %, still more preferably from about 5 wt. % to about 20 wt. %, based on the total weight of the final, stabilized product. If desired, more amphoteric polymer can be added to reduce the moisture of the final product. However, because the amphoteric polymer is relatively expensive, the composition of the final product can be modified using less expensive absorbents, bulking agents and/or flowing agents, as described further below.

The infusion method of the invention produces a generally-solid, stabilized product with several advantages. First, the generally-solid product avoids the need to use heavy liquid ingredients in the manufacture of the final food or pharmaceutical preparation. The generally-solid product is also easy to weigh out and mix, does not need to be refrigerated, and disperses easily in a solution. Accordingly, there is no need to use a homogenizer to introduce the product into a beverage. The flavor, color or other active ingredient is protected from oxidation and heat degradation, so that the true flavor or color profile is maintained. The flavor, color or other active ingredient is also protected from mechanically-forced oozing out, which results, for example, when liquid mint oil is used in a tablet press. The stabilized product also permits thermally-controlled release of the active ingredient, which is beneficial, for example, for spice blends in heated pizza sauce, garlic or onion flavor in bakery goods, or lemon flavor in hot tea. The inventive infusion method prolongs the shelf stability of the active ingredient. It also increases the content of the active ingredient in solid form compared to existing methods. For example, mint flavor powders produced in accordance with the present invention can have a content up to 50%, versus those produced by prior art methods, which could only have a content of about 20%.

Where the liquid to be entrapped or infused is not water soluble, for example, an oleoresin, preferably an emulsifier is mixed with the liquid and amphoteric polymer to enhance the organic affinity of the amphoteric polymer and to make the amphoteric polymer more hydrophilic. Particularly preferred emulsifiers suitable for use in the present invention include polyoxyethylene sorbitan fatty acid esters, such as Tween 20 (laurate), Tween 21 (laurate), Tween 40 (palmitate), Tween 60 (stearate), Tween 65 (tri-stearate), Tween 80 (oleate), Tween 81 (oleate) and Tween 85 (tri-oleate), and mixtures thereof. Tween products are commercially available from SPI Polyols Inc. (New Castle, Del.).

If desired, although not necessary, certain oil-based flavors can be dissolved in a solvent before addition of the amphoteric polymer. Examples of solvents suitable for use in the present invention include water, ethyl alcohol, propylene glycol, triacetine, glycerine, triglyceride, corn oil, soy bean oil, canola oil, triethyl citrate, sunflower oil, and coconut oil.

Preferably an absorbent is also mixed with the liquid and amphoteric polymer. The absorbent attracts and holds water, and advantageously attracts free liquid from the powder. Absorbents generally include agents that are porous and have a sufficiently large surface area so that they can absorb liquid into their matrix.

Suitable absorbents useful in connection with the present invention include water-soluble polymers, and thickening gums. Specific examples of useful water soluble polymers include cellulose and cellulose derivatives, including alkylcelluloses, hydroxyalkylcelluloses and hydroxyalkylalkylcelluloses, such as methyl cellulose, hydroxypropylmethyl cellulose, hydroxyethyl cellulose, hydroxypropyl cellulose, carboxymethyl cellulose, hydroxyethylmethylcellulose, hydroxypropylmethylcellulose, hydroxybutylmethylcellulose, cellulose esters and cellulose ester derivatives, such as cellulose acetate phthalate (CAP), hydroxypropylmethylcellulose (HPMC), carboxyalkylcelluloses, carboxyalkylalkylcelluloses, carboxyalkylcellulose esters such as carboxymethylcellulose, and their alkali metal salts. Other water soluble polymers include polyvinyl alcohol, sodium alginate, polyethylene glycol, xanthan gum, tragancanth, guar gum, acacia gum, polyacrylic acid, methylmethacrylate copolymers, carboxyvinyl copolymers, calcium silicate, silicon dioxide and combinations thereof. Other polymers useful as absorbents for incorporation into the films of the present invention include biodegradable polymers, copolymers, block polymers and combinations thereof. Examples of such polymers that meet the above criteria include poly(glycolic acid) (PGA), poly(lactic acid) (PLA), polydioxanoes, polyoxalates, poly(α-esters), polyanhydrides, polyacetates, polycaprolactones, poly(orthoesters), polyamino acids, polyurethanes, polycarbonates, polyaminocarbonates, polyamides, poly(alkyl cyanoacrylates), and mixtures and copolymers thereof. Additional useful poymers include stereopolymers of L- and D-lactic acid, copolymers of bis(p-carboxyphenoxy) propane acid and sebacic acid, sebacic acid copolymers, copolymers of caprolactone, poly(lactic acid)/poly(glycolic acid)/polyethyleneglycol copolymers, copolymers of polyurethane and (poly(lactic acid), copolymers of polyurethane and poly(lactic acid), copolymers of α-amino acids, copolymers of α-amino acids and capric acid, copolymers of α-benzyl glutamate and polyethylene glycol, copolymers of succinate and poly(glycols), polyphosphazene, polyhydroxy-alkanoates and mixtures thereof. Binary and ternary systems are also contemplated as absorbents. Other useful absorbents include, for example, gelatin, vegetable proteins such as sunflower protein, soybean proteins, cotton seed proteins, peanut proteins, grape seed proteins, whey proteins, whey protein isolates, blood proteins, egg proteins, acrylated proteins, water-soluble polysaccharides such as alginates, carrageenans, guar gum, agar-agar, xanthan gum, gellan gum, gum arabic and related gums (such as gum ghatti, gum karaya, and gum tragacanth), pectin, water-soluble derivatives of water-soluble synthetic polymers such as polyacrylic acids and polyacrylic acid esters, polymethacrylic acids and polymethacrylic acid esters, polyvinylacetates, polyvinylalcohols, polyvinylacetatephthalates (PVAP), PVY/vinyl acetate copolymers, and polycrotonic acids. Also suitable are phthalated gelatin, gelatin succinate, crosslinked gelatin, shellac, water soluble chemical derivatives of starch, cationically modified acrylates and methacrylates possessing, for example, a tertiary or quaternary amino group, such as the diethylaminoethyl group, which may be quaternized if desired.

The absorbent is preferably included in an amount ranging from about 1 wt. % to about 70 wt. %, more preferably from about 4 wt. % to about 50 wt. %, still more preferably from about 5 wt. % to about 30 wt. %.

In a particularly preferred embodiment, a flowing agent is combined with the liquid and amphoteric polymer to form a free-flowing mixture to prevent or minimize the electrostatic effect and enhance the flow of the final product, which is in the form of a product. Examples of suitable flowing agents useful in connection with the invention are silicon dioxide and calcium silicate. The flowing agent is preferably included in an amount ranging from about 1 wt. % to about 20 wt. %, more preferably from about 5 wt. % to about 10 wt. %.

If desired, the mixture can further include an inert bulking agent. Bulking agents are economically desirable, as they increase the volume of the final product without substantial cost. Suitable inert bulking agents include lactose, starches (such as rice, corn and potato starches), hydrolyzed guar gum (such as BENEFIBER®, commercially available from Novartis Nutrition, Minneapolis, Minn.), and hydrolyzed vegetable gum. The bulking agent is preferably included in an amount ranging from about 1 wt. % to about 80 wt. %, more preferably from about 10 wt. % to about 70 wt. %, still more preferably from about 30 wt. % to about 60 wt. %.

In order to stabilize the food-grade or pharmaceutical-grade liquid, preferably the liquid is first mixed with any emulsifier(s) and/or solvent(s) until a homogeneous solution is achieved. The amphoteric polymer is then added to the solution until a dough-like paste is obtained. Thereafter, any absorbents, flowing agents and/or bulking agents are added, preferably with the absorbent(s) added first and the flowing agent(s) added last. The methods of the invention are performed in the absence of heat, and preferably at room temperature.

### EXAMPLES

The following examples describe the stabilization of food-grade or pharmaceutical-grade liquids.

### Example 1

### Color Stabilization

Oleoresin paprika (commercially available from SpiceTec-USF, Carol Stream, Ill.) is mixed with Tween 80 until a homogeneous solution is obtained, and PVP is mixed with the solution until a dough-like paste is achieved. Thereafter modified cellulose is added to the paste under a brief high-shear mixer to break any lumps that may have occurred. Then food starch is added under a mild stir until a uniform powder is produced. The final powder product is stored in an air-tight container until use. The amounts of the components are shown in Table 1 and expressed as weight percents based on the total weight of the final stabilized paprika product.

TABLE 1

| | |
|---|---|
| Oleoresin paprika | 10 wt. % |
| Tween 80 | 5 wt. % |
| PVP | 8 wt. % |
| Modified cellulose | 7 wt. % |
| Food starch | 70 wt. % |

### Example 2

### Color Stabilization

Oleoresin capsicum (commercially available from SpiceTec-USF, Carol Stream, Ill.) is mixed with Tween 60 until a homogeneous solution is obtained, and PVP is mixed with the solution until a dough-like paste is achieved. Thereafter calcium silicate is added to the paste under a brief high-shear mixer to break any lumps that may have occurred. Then food starch is added under a mild stir until a uniform powder is produced. The final powder product is stored in an air-tight container until use. The amounts of the components are shown in Table 2 and expressed as weight percents based on the total weight of the final stabilized oleoresin product.

TABLE 2

| | | |
|---|---|---|
| Oleoresin capsicum | 25 | wt. % |
| Tween 60 | 30 | wt. % |
| PVP | 20 | wt. % |
| Food Starch | 7.5 | wt. % |
| Calcium silicate | 17.5 | wt. % |

### Example 3

### Color Stabilization

PVP is mixed with black pepper oleoresin (commercially available from SpiceTec-USF, Carol Stream, Ill.) until a dough-like paste is achieved. Thereafter modified cellulose is added to the paste under a brief high-shear mixer to break any lumps that may have occurred. Then food starch is added under a mild stir until a uniform powder is produced. The final powder product is stored in an air-tight container until use. The amounts of the components are shown in Table 3 and expressed as weight percents based on the total weight of the final stabilized color product.

TABLE 3

| | |
|---|---|
| Black pepper oleoresin | 15 wt. % |
| PVP | 5 wt. % |
| Starch | 60 wt. % |
| Modified cellulose | 60 wt. % |

### Example 4

### Color Stabilization

Lycopen extract oil (15% from tomato paste) (commercially available from Kalsec, Inc., Kalamazoo, Mich.) is combined with propylene glycol and Tween 60 until a homogeneous solution is obtained, and PVP is mixed with the solution until a dough-like paste is achieved. Thereafter modified cellulose and oligo fructose are added to the paste under a brief high-shear mixer to break any lumps that may have occurred. Then lactose monohydrate is added under a mild stir until a uniform powder is produced. The final powder product is stored in an air-tight container until use. The amounts of the components are shown in Table 4 and expressed as weight percents based on the total weight of the final stabilized oleoresin product.

TABLE 4

| | |
|---|---|
| Lycopen extract oil | 16 wt. % |
| Tween 60 | 5 wt. % |
| Propylene glycol | 3 wt. % |
| PVP | 8 wt. % |
| Modified cellulose | 20 wt. % |
| Oligo fructose | 32 wt. % |
| Lactose monohydrate | 16 wt. % |

### Example 5

### Volatile Essence Stabilization

Diacetyl flavor (commercially available from Fleurchem, Inc., Middletown, N.Y.) is mixed with propylene glycol until a homogeneous solution is obtained, and PVP is mixed with the solution until a dough-like paste is achieved. Thereafter food starch is added under a mild stir until a uniform powder is produced. The final powder product is stored in an air-tight container until use. The amounts of the components are shown in Table 5 and expressed as weight percents based on the total weight of the final stabilized oleoresin product.

TABLE 5

| | | |
|---|---|---|
| Diacetyl flavor | 24 | wt. % |
| Propylene glycol | 3.5 | wt. % |
| PVP | 8 | wt. % |
| Starch | 64.5 | wt. % |

### Example 6

### Volatile Essence Stabilization

PVP is mixed with acetaldehyde (50% in ethanol) (commercially available from Advanced BioTech, Visalia, Calif.) until a dough-like paste is achieved. Thereafter oligo fructose is added to the paste under a brief high-shear mixer to break any lumps that may have occurred. Then α-lactose is added under a mild stir until a uniform powder is produced. The final powder product is stored in an air-tight container until use. The amounts of the components are shown in Table 6 and expressed as weight percents based on the total weight of the final stabilized color product.

TABLE 6

| | |
|---|---|
| Acetaldehyde (50% in | 5 wt. % |
| ethanol) | |
| PVP | 5 wt. % |
| Oligo fructose | 50 wt. % |
| α-Lactose | 40 wt. % |

### Example 7

### Volatile Essence Stabilization

PVP is mixed with natural mustard flavor (commercially available from Kalsec, Inc., Kalamazoo, Mich.) until a dough-like paste is achieved. Thereafter modified cellulose is added to the paste under a brief high-shear mixer to break any lumps that may have occurred. Then food starch is added under a mild stir until a uniform powder is produced. The final powder product is stored in an air-tight container until use. The amounts of the components are shown in Table 7 and expressed as weight percents based on the total weight of the final stabilized color product.

TABLE 7

| | |
|---|---|
| Natural mustard flavor | 27 wt. % |
| PVP | 10 wt. % |
| Modified cellulose | 17 wt. % |
| Starch | 40 wt. % |

### Example 8

### Volatile Essence Stabilization

PVP is mixed with peppermint oil (commercially available from Citrus & Allied Essences, Ltd., Lake Success, N.Y.) until a dough-like paste is achieved. Thereafter modified cellulose is added to the paste under a brief high-shear mixer to break any lumps that may have occurred. Then hydrolyzed vegetable gum is added under a mild stir until a uniform powder is produced. The final powder product is stored in an air-tight container until use. The amounts of the components are shown in Table 8 and expressed as weight percents based on the total weight of the final stabilized color product.

TABLE 8

| | |
|---|---|
| Peppermint oil | 20 wt. % |
| PVP | 20 wt. % |
| Modified cellulose | 18 wt. % |
| Hydrolyzed vegetable gum | 42 wt. % |

### Example 9

### Volatile Essence Stabilization

PVP is mixed with lemon flavor (commercially available from Treatt PLC, Haines City, Fla.) until a dough-like paste is achieved. Thereafter modified cellulose is added to the paste under a brief high-shear mixer to break any lumps that may have occurred. Then food starch is added under a mild stir until a uniform powder is produced. The final powder product is stored in an air-tight container until use. The amounts of the components are shown in Table 9 and expressed as weight percents based on the total weight of the final stabilized color product.

TABLE 9

| | | |
|---|---|---|
| Lemon flavor | 24 | wt. % |
| PVP | 8 | wt. % |
| Modified cellulose | 1.5 | wt. % |
| Starch | 66.5 | wt. % |

### Example 10

### Volatile Essence Stabilization

PVP is mixed with butter flavor (commercially available from Advanced BioTech, Visalia, Calif.) until a dough-like paste is achieved. Thereafter wood fiber is mixed with the paste, and then modified cellulose is added to the paste under a brief high-shear mixer to break any lumps that may have occurred. Then α-lactose is added under a mild stir until a uniform powder is produced. The final powder product is stored in an air-tight container until use. The amounts of the components are shown in Table 10 and expressed as weight percents based on the total weight of the final stabilized color product.

TABLE 10

| | |
|---|---|
| Butter flavor | 10 wt. % |
| PVP | 7 wt. % |
| Modified cellulose | 25 wt. % |
| Wood fiber | 12 wt. % |
| α-Lactose | 46 wt. % |

The preceding description has been presented with reference to presently preferred embodiments of the invention. Workers skilled in the art and technology to which this invention pertains will appreciate that alterations and changes in the described methods and compositions may be practiced without meaningfully departing from the principal, spirit and scope of this invention. Accordingly, the foregoing description should not be read as pertaining only to the precise methods and compositions described, but rather should be read consistent with and as support to the following claims which are to have their fullest and fair scope.

1. A method for increasing the stability of a food-grade or pharmaceutical-grade liquid comprising mixing the liquid with an amphoteric polymer to thereby infuse the liquid into the amphoteric polymer matrix and form a generally-solid, stabilized product.

2. The method of claim 1, wherein the food-grade or pharmaceutical-grade liquid is selected from the group consisting of extracted oils, herbal extracts, flavors, colors, and volatile chemical components used in the flavor industry.

3. The method of claim 1, wherein the food-grade or pharmaceutical-grade liquid is a natural or synthetic flavoring liquid.

4. The method of claim 1, wherein the food-grade or pharmaceutical-grade liquid is selected from the group consisting of volatile oils, synthetic flavor oils, flavoring aromatics, oleoresins, extracts derived from plants, leaves flowers, fruits and stems, and combinations thereof.

5. The method of claim 1, wherein the food-grade or pharmaceutical-grade liquid is selected from the group consisting of citrus oils, lime and grapefruit oils, and fruit essences.

6. The method of claim 1, wherein the food-grade or pharmaceutical-grade liquid is selected from the group consisting of aldehydes and esters.

7. The method of claim 1, wherein the food-grade or pharmaceutical-grade liquid is selected from the group consisting of benzaldehyde, citral, neral, decanal, aldehyde C-9, aldehyde C-12, tolyl aldehyde, 2,6-dimethyloctanol, 2-dodecenal, and combinations thereof.

8. The method of claim 1, wherein the food-grade or pharmaceutical-grade liquid is selected from the group consisting of mint flavor, diacetyl flavor, mustard flavor, peppermint oil, lemon flavor, cheese flavor, acetaldehyde, onion oil, garlic oil, mustard flavor, smoke flavor, honey flavor, maple flavor, yeast dough flavor, cilantro flavor, tabasco flavor, worcestershire flavor, coffee flavor, and coconut flavor.

9. The method of claim 1, wherein the food-grade or pharmaceutical-grade liquid is selected from the group consisting of paprika, capsicum, black pepper, apsicum, basil, oregano, ginger, clove, curry blend, jalapeno, chipotle chili, cajun blend, cilantro, celery, chili spice, green bell pepper, barbecue blend allspice, and chili ancho oleoresins.

10. The method of claim 1, wherein the food-grade or pharmaceutical-grade liquid is selected from the group consisting of dimethyl sulfide, isobutyl aldehyde, 2-methyl-3-furanthiol, methional, acetaldehyde and sulfural.

11. The method of claim 1, wherein the food-grade or pharmaceutical-grade liquid is included in an amount ranging from about 3 wt. % to about 70 wt. %, based on the total weight of the stabilized product.

12. The method of claim 1, wherein the food-grade or pharmaceutical-grade liquid is included in an amount ranging from about 4 wt. % to about 50 wt. %, based on the total weight of the stabilized product.

13. The method of claim 1, wherein the food-grade or pharmaceutical-grade liquid is included in an amount ranging from about 5 wt. % to about 30 wt. %, based on the total weight of the stabilized product.

14. The method of claim 1, wherein the amphoteric polymer is polyvinylpyrrolidone.

15. The method of claim 1, wherein the amphoteric polymer is included in an amount ranging from about 3 wt. % to about 40 wt. %, based on the total weight of the stabilized product.

16. The method of claim 1, wherein the amphoteric polymer is included in an amount ranging from about 4 wt. % to about 30 wt. %, based on the total weight of the stabilized product.

17. The method of claim 1, wherein the amphoteric polymer is included in an amount ranging from about 5 wt. % to about 20 wt. %, based on the total weight of the stabilized product.

18. The method of claim 1, further comprising mixing an emulsifier with the liquid prior to introduction of the amphoteric polymer.

19. The method of claim 18, wherein the emulsifier is selected from the group consisting of Tween 20, Tween 21, Tween 40, Tween 60, Tween 65, Tween 80, Tween 81, Tween 85 and mixtures thereof.

20. The method of claim 1, further comprising mixing an absorbent with the liquid and amphoteric polymer.

21. The method of claim 20, wherein the absorbent is selected from the group consisting of water-soluble polymers and thickening gums.

22. The method of claim 20, wherein the absorbent is selected from the group consisting of cellulose, cellulose derivatives, cellulose esters and cellulose ester derivatives.

23. The method of claim 20, wherein the absorbent is selected from the group consisting of polyvinyl alcohol, sodium alginate, polyethylene glycol, xanthan gum, tragancanth, guar gum, acacia gum, polyacrylic acid, methylmethacrylate copolymers, carboxyvinyl copolymers, calcium silicate, silicon dioxide and combinations thereof.

24. The method of claim 20, wherein the absorbent is selected from the group consisting of poly(glycolic acid) (PGA), poly(lactic acid) (PLA), polydioxanoes, polyoxalates, poly(α-esters), polyanhydrides, polyacetates, polycaprolactones, poly(orthoesters), polyamino acids, polyurethanes, polycarbonates, polyaminocarbonates, polyamides, poly(alkyl cyanoacrylates), stereopolymers of L- and D-lactic acid, copolymers of bis(p-carboxyphenoxy) propane acid and sebacic acid, sebacic acid copolymers, copolymers of caprolactone, poly(lactic acid)/poly(glycolic acid)/polyethyleneglycol copolymers, copolymers of polyurethane and poly(lactic acid), copolymers of polyurethane and poly(lactic acid), copolymers of α-amino acids, copolymers of α-amino acids and caproic acid, copolymers of α-benzyl glutamate and polyethylene glycol, copolymers of succinate and poly(glycols), polyphosphazene, polyhydroxy-alkanoates and mixtures thereof.

25. The method of claim 20, wherein the absorbent is selected from the group consisting of gelatin, vegetable proteins, soybean proteins, cotton seed proteins, peanut proteins, grape seed proteins, whey proteins, whey protein isolates, blood proteins, egg proteins, acrylated proteins, water-soluble polysaccharides, carrageenans, guar gum, agar-agar, xanthan gum, gellan gum, gum arabic, gum ghatti, gum karaya, gum tragacanth, pectin, phthalated gelatin, gelatin succinate, crosslinked gelatin, shellac, water soluble chemical derivatives of starch, and combinations thereof.

26. The method of claim 1, further comprising mixing an inert bulking agent with the liquid and amphoteric polymer.

27. The method of claim 1, further comprising mixing a flowing agent with the liquid and amphoteric polymer.

28. The method of claim 1, wherein the method is performed in the absence of heat.

29. The method of claim 1, wherein the method is performed at room temperature.

30. The method of claim 1, further comprising:
mixing an emulsifier with the liquid prior to introduction of the amphoteric polymer;
mixing an absorbent with the liquid and amphoteric polymer; and
mixing an inert bulking agent with the liquid and amphoteric polymer.

31. The method of claim 30, wherein the amphoteric polymer is polyvinylpyrrolidone.

32. A stabilized generally-solid composition comprising an amphoteric polymer in which is infused a food-grade or pharmaceutical-grade liquid.

33. The composition of claim 32, wherein the food-grade or pharmaceutical-grade liquid is selected from the group consisting of extracted oils, herbal extracts, flavors, colors, and volatile chemical components used in the flavor industry.

34. The composition of claim 32, wherein the food-grade or pharmaceutical-grade liquid is present in an amount ranging from about 3 wt. % to about 70 wt. %, based on the total weight of the composition.

35. The composition of claim 32, wherein the amphoteric polymer is polyvinylpyrrolidone.

36. The composition of claim 32, wherein the amphoteric polymer is present in an amount ranging from about 3 wt. % to about 40 wt. %, based on the total weight of the composition.

37. The composition of claim 32, further comprising an emulsifier.

38. The composition of claim 32, further comprising an absorbent.

39. The composition of claim 32, further comprising an inert bulking agent.

40. The composition of claim 32, further comprising a flowing agent.
